SEQUENCE LISTING (36 sequences)

SEQ ID NO: 1 - DNA, 420 bp, artificial sequence
  CKX fragment
cctcatccctggctcaacgtgctcgtgccccgctccggcatcgccgacttcgaccgcgccgtcttcaggggcatcctccagggcaccgacatcgccgggcccctcgtcgtctacccactcaacaaatccaagtgggacgacggcatgtcggcggtgacgccggcggaggaggtgttctacgcggtgtcgatgctcttctcgtcggtggccaacgacctgaggcggctggaggcgcagaaccagaagatactgcggttctgcgacctcgccgggatagggtacaaggagtacctggcgcactacacggcccacggcgactgggtccggcacttcggcggcaagtggaagcacttcgtggagatgaaggacaagtacgaccccaagaagctgctctccccgggccaagacatcttcaactaa

SEQ ID NO: 2 - DNA, 1828 bp, artificial sequence
  CKX fragment
agtgaaccactaccctgctacacgcctttcatctttctcctgaaaacctcaccactaaacttatttattcttgacctacagaagagccatgaggcaattactcctgcaatacctgaagctgttcctgttgctaggccttggcgcagtcactgctgagcatgtgcttaaacatgatgtgcttgcatccctggggacgctcccccttgacgggcatttcagcttccacgacttgtctgcagctgcaatggacttcggcaacctctctagcttcccgccagtcgctgtgcttcacccaggttcagtggctgacattgccacaaccgtgaggcatgtgttcttgatgggtgagcactccgcgctcacagtggcagctcgtgggcatggacactcgctatatgggcagtcccaggctgctggagggattgtcatcagaatggaatcccttcggagtgtcaaaatgcaggtgcatcctggtgcatcaccctatgtggatgcctcaggaggcgaactctggataaatgtcttgaataagacgttgaagtatggtttggcgccgaagtcatggacagactacctccaccttacggttgggggcacgttgtcaaatgcgggcgtcagcgggcagacattccggcatggtccacagatcagcaatgtgaacgaattggagattgtgactggaagaggtgatattgtcacttgctcaccagaacagaactctgatctcttccgtgctgctcttggtggtctgggtcagtttggcatcattactcgggccaggatcgcacttgagcctgctccacaaatggtgaggtggataagagttctctacttagatttcatgagcttcaccgaggatcaggagatgcttatttcagcagagaagaccttcgactacattgaaggtttcgttatcataaacagaacaggcatcctaaacaactggaggtcatcgttcaatccacaggacccagagcgggctagccggttcgaaacagacagaaaagtgctcttctgcctcgagatgacaaagaacttcaaccctgaagaagctgacatcatggaacaggaggtccatgcactactatctcaacttagatacacaccagcctccttattccacacggacgtcacttacattgagttcttggatagggtgcactcctctgagatgaagctgagagctaagggcttgtgggaagtcccacacccatggcttaatctcatcataccaagaagcactatccatacatttgcagagcaggtctttgggaaaatcctcgaagataacaacaatggtcccatattgctctacccagtgaagaagtccagatgggacaaccgaacgtcagtggtcataccagatgaggaagttttctacctggtgggattcctatcctcggcgataggcccccacagcatcgaacatacattgaacctgaacaaccagataatagagttctctaacaaagcaagtattggggtgaagcaatatcttccaaactacaccacagaacccgagtggaaggcccactatggggctaggtgggacgcatttcaacagaggaaaaacacctatgaccccctggcaatcctagctccaggacagaaaatatttcaaaagaaaccagcatcactacccttgtcctcgttacagtacctactgtaaaaaatatatatgtggagcaatatgtctatgttagtatggaactatagtcgctttgcaaaagataacgaactgcagcgtgaaggacactgtacagagtagtgactattagtagtggtgatgctcaaaatacttttagcactgagatcaatgaagatcagc

SEQ ID NO: 3 - DNA, 789 bp, artificial sequence
  CKX fragment
ggccgccgggtgaggaactcgggcgggttcttcaccgacgccgacgtcgccaggatcgtggccgtcgccgcggcgaggaacgctaccaccgtgtacgtcatcgagacgacgctcaactacgacagcgccacggccgcgtccgtggaccaggagctcagctcggtgctggcgacgctgaggcacgaggaggggcacgcgttcgtgcgggacgcgtcgtacctggagttcctggaccgggtgcacggcgaggagggggcgctggacaagatcgggctgtggcgtgtcccgcacccttggctcatcgtgctcgtgccccgctcccgcatcgccgacttcgaccgcggcgtcttcaagggcatcctccagggcaccgacatcgccgggcctctcgtcgtctacccgctcaacaaatccaagtacgcactacgcgtccatcgtgcatgcatgcatgcgatcctgcatggtatgtatcacgcacttaactgacactggctcggtcgtggtttcaggtgggacgacggcatgtcggcggtgacgccagcggaggaggtgttctacgcggtgtcgctgcccttctcgtcggtggccaacgacctgaagcggctggaggcgcagaaccagaagatactgcggttctgcgacctcgccggggtagggtacaaggagtacctggcgcactacacggcccacggcgactgggtccggcacttcggcggcaagtggcagcgcttcgtggagatgaaggacaagtacgaccccaagaggctgctctccccaggccaggacatc

SEQ ID NO: 4 - DNA, 585 bp, artificial sequence
  TaCKX4 fragment
gctgtctcggctgagatacatacagtcaaccctcttccacaccgacgtcacgtacctcgagttcctggacagggtgcactcctccgagctcaagctcagggcccagggcctctgggaggttccacacccatggctcaacctcctcatcccgagaagcaccatccaccggttcgcgcgggaggtcttcggcaagatcctgaaagacagcaacaatggccccatactcctctacccagtaaacagatcaaagtgggacaacaggacgtcagtggtgataccggaagaagaaatcttctacctggtggggttcctgtcgtcggcaccgtccgcctcaggccacggcagcgtcgagcatgcggtgagcctgaacgacaagatcgtagacttctgcgacaaggcgggcgtcgggatgaagcagtacctagcgccctacaccacccagcagcagtggaaagcccacttcggggcgaggtgggagacatttgagcggaggaaacacatgtatgatcccctagcaatcctagccccaggacagagaatatttccaaaggcatcactgcccatgtcctcttgacaaacagcatc

SEQ ID NO: 5 - DNA, 538 bp, artificial sequence
  TaCKX7 fragment
cgtgcaggacgtgtcgtacgcggggttccttgaccgggtgcgcgacggcgagctcaagctccgcgccgccggcctctgggacgtgccgcacccctggctcaacctcttcctcccgcgctcccgcgtcctcgacttcgcggccggcgtcttccacggcatcctccgccgcgacggcggcaccggccccatgggccccgtcctcgtctaccccatgaaccgggacaggtgggacggcaacacgtcagcggtgttcccagaggaggaggaggtgttctacacggttgggatccttcggtcggcggtgtccgagggtgaccttgggcgtctggaggagcagaacgaggagatcttacgcttctgcgaggaggcagggataccgtgcgtgcagtacctgccgtactacgccgaccaggccgggtgggagaagaagcactttggtccagccaagtgggccaggttcgtggagcggaagagggagtatgaccccaaggcgatcctatcccgtggtcagagaattttcacctccccgctggcttga

SEQ ID NO: 6 - DNA, 4697 bp, artificial sequence
  silencing cassette for gene HvCKX1
aatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccctaacaagcccaccaaagcaaaaagcccactgctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcccccatttaaatcccccctcgaggatcaagtgcaaaggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatgtcttttatatccttcactaccatgaaaagactagctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcggccatgggagggcgcgccagagatatcagacggaccgcctcatccctggctcaacgtgctcgtgccccgctccggcatcgccgacttcgaccgcgccgtcttcaggggcatcctccagggcaccgacatcgccgggcccctcgtcgtctacccactcaacaaatccaagtgggacgacggcatgtcggcggtgacgccggcggaggaggtgttctacgcggtgtcgatgctcttctcgtcggtggccaacgacctgaggcggctggaggcgcagaaccagaagatactgcggttctgcgacctcgccgggatagggtacaaggagtacctggcgcactacacggcccacggcgactgggtccggcacttcggcggcaagtggaagcacttcgtggagatgaaggacaagtacgaccccaagaagctgctctccccgggccaagacatcttcaactaaagacctaggaaggtatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtggagctcagagcgatcgcagacccgggaggttagttgaagatgtcttggcccggggagagcagcttcttggggtcgtacttgtccttcatctccacgaagtgcttccacttgccgccgaagtgccggacccagtcgccgtgggccgtgtagtgcgccaggtactccttgtaccctatcccggcgaggtcgcagaaccgcagtatcttctggttctgcgcctccagccgcctcaggtcgttggccaccgacgagaagagcatcgacaccgcgtagaacacctcctccgccggcgtcaccgccgacatgccgtcgtcccacttggatttgttgagtgggtagacgacgaggggcccggcgatgtcggtgccctggaggatgcccctgaagacggcgcggtcgaagtcggcgatgccggagcggggcacgagcacgttgagccagggatgaggactagtgggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcagga

SEQ ID NO: 7 - DNA, 4467 bp, artificial sequence
  silencing cassette for gene HvCKX2
aatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccctaacaagcccaccaaagcaaaaagcccactgctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcccccatttaaatcccccctcgaggatcaagtgcaaaggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatgtcttttatatccttcactaccatgaaaagactagctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcggccatgggagggcgcgccagagatatcagacggaccgactagtgaggcgaactctggataaatgtcttgaataagacgttgaagtatggtttggcgccgaagtcatggacagactacctccaccttacggttgggggcacgttgtcaaatgcgggcgtcagcgggcagacattccggcatggtccacagatcagcaatgtgaacgaattggagattgtgactggaagaggtgatattgtcacttgctcaccagaacagaactctgatctcttccgtgctgctcttggtggtctgggtcagtttgagctccctaggaaggtatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtggagctcaaactgacccagaccaccaagagcagcacggaagagatcagagttctgttctggtgagcaagtgacaatatcacctcttccagtcacaatctccaattcgttcacattgctgatctgtggaccatgccggaatgtctgcccgctgacgcccgcatttgacaacgtgcccccaaccgtaaggtggaggtagtctgtccatgacttcggcgccaaaccatacttcaacgtcttattcaagacatttatccagagttcgcctcactagtgggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttc

SEQ ID NO: 8 - DNA, 5431 bp, artificial sequence
  silencing cassette for gene TaCKX1
aatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccctaacaagcccaccaaagcaaaaagcccactgctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcccccatttaaatcccccctcgaggatcaagtgcaaaggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatgtcttttatatccttcactaccatgaaaagactagctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcggccatgggagggcgcgccagagatatcagacggaccgggccgccgggtgaggaactcgggcgggttcttcaccgacgccgacgtcgccaggatcgtggccgtcgccgcggcgaggaacgctaccaccgtgtacgtcatcgagacgacgctcaactacgacagcgccacggccgcgtccgtggaccaggagctcagctcggtgctggcgacgctgaggcacgaggaggggcacgcgttcgtgcgggacgcgtcgtacctggagttcctggaccgggtgcacggcgaggagggggcgctggacaagatcgggctgtggcgtgtcccgcacccttggctcatcgtgctcgtgccccgctcccgcatcgccgacttcgaccgcggcgtcttcaagggcatcctccagggcaccgacatcgccgggcctctcgtcgtctacccgctcaacaaatccaagtacgcactacgcgtccatcgtgcatgcatgcatgcgatcctgcatggtatgtatcacgcacttaactgacactggctcggtcgtggtttcaggtgggacgacggcatgtcggcggtgacgccagcggaggaggtgttctacgcggtgtcgctgcccttctcgtcggtggccaacgacctgaagcggctggaggcgcagaaccagaagatactgcggttctgcgacctcgccggggtagggtacaaggagtacctggcgcactacacggcccacggcgactgggtccggcacttcggcggcaagtggcagcgcttcgtggagatgaaggacaagtacgaccccaagaggctgctctccccaggccaggacatccctaggaaggtatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtggagctcagagcgatcgcagacccggggatgtcctggcctggggagagcagcctcttggggtcgtacttgtccttcatctccacgaagcgctgccacttgccgccgaagtgccggacccagtcgccgtgggccgtgtagtgcgccaggtactccttgtaccctaccccggcgaggtcgcagaaccgcagtatcttctggttctgcgcctccagccgcttcaggtcgttggccaccgacgagaagggcagcgacaccgcgtagaacacctcctccgctggcgtcaccgccgacatgccgtcgtcccacctgaaaccacgaccgagccagtgtcagttaagtgcgtgatacataccatgcaggatcgcatgcatgcatgcacgatggacgcgtagtgcgtacttggatttgttgagcgggtagacgacgagaggcccggcgatgtcggtgccctggaggatgcccttgaagacgccgcggtcgaagtcggcgatgcgggagcggggcacgagcacgatgagccaagggtgcgggacacgccacagcccgatcttgtccagcgccccctcctcgccgtgcacccggtccaggaactccaggtacgacgcgtcccgcacgaacgcgtgcccctcctcgtgcctcagcgtcgccagcaccgagctgagctcctggtccacggacgcggccgtggcgctgtcgtagttgagcgtcgtctcgatgacgtacacggtggtagcgttcctcgccgcggcgacggccacgatcctggcgacgtcggcgtcggtgaagaacccgcccgagttcctcacccggcggccactagtgggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacc

SEQ ID NO: 9 - DNA, 8172 bp, artificial sequence
  T-DNA region of vector pMCG/HvCKX1
aattctatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagtgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcactagagtcgaccctagtgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatctagggcatgcgggggttaattaacccccgaattccaatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccctaacaagcccaccaaagcaaaaagcccactgctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcccccatttaaatcccccctcgaggatcaagtgcaaaggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatgtcttttatatccttcactaccatgaaaagactagctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcggccatgggagggcgcgccagagatatcagacggaccgcctcatccctggctcaacgtgctcgtgccccgctccggcatcgccgacttcgaccgcgccgtcttcaggggcatcctccagggcaccgacatcgccgggcccctcgtcgtctacccactcaacaaatccaagtgggacgacggcatgtcggcggtgacgccggcggaggaggtgttctacgcggtgtcgatgctcttctcgtcggtggccaacgacctgaggcggctggaggcgcagaaccagaagatactgcggttctgcgacctcgccgggatagggtacaaggagtacctggcgcactacacggcccacggcgactgggtccggcacttcggcggcaagtggaagcacttcgtggagatgaaggacaagtacgaccccaagaagctgctctccccgggccaagacatcttcaactaaagacctaggaaggtatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtggagctcagagcgatcgcagacccgggaggttagttgaagatgtcttggcccggggagagcagcttcttggggtcgtacttgtccttcatctccacgaagtgcttccacttgccgccgaagtgccggacccagtcgccgtgggccgtgtagtgcgccaggtactccttgtaccctatcccggcgaggtcgcagaaccgcagtatcttctggttctgcgcctccagccgcctcaggtcgttggccaccgacgagaagagcatcgacaccgcgtagaacacctcctccgccggcgtcaccgccgacatgccgtcgtcccacttggatttgttgagtgggtagacgacgaggggcccggcgatgtcggtgccctggaggatgcccctgaagacggcgcggtcgaagtcggcgatgccggagcggggcacgagcacgttgagccagggatgaggactagtgggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcagga

SEQ ID NO: 10 - DNA, 414 bp, artificial sequence
  sequence of the inserted HvCKX1 gene fragment, sense orientation
atccctggctcaacgtgctcgtgccccgctccggcatcgccgacttcgaccgcgccgtcttcaggggcatcctccagggcaccgacatcgccgggcccctcgtcgtctacccactcaacaaatccaagtgggacgacggcatgtcggcggtgacgccggcggaggaggtgttctacgcggtgtcgatgctcttctcgtcggtggccaacgacctgaggcggctggaggcgcagaaccagaagatactgcggttctgcgacctcgccgggatagggtacaaggagtacctggcgcactacacggcccacggcgactgggtccggcacttcggcggcaagtggaagcacttcgtggagatgaaggacaagtacgaccccaagaagctgctctccccgggccaagacatcttcaact

SEQ ID NO: 11 - DNA, 7942 bp, artificial sequence
  T-DNA region of vector pMCG/HvCKX2
aattctatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagtgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcactagagtcgaccctagtgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatctagggcatgcgggggttaattaacccccgaattccaatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccctaacaagcccaccaaagcaaaaagcccactgctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcccccatttaaatcccccctcgaggatcaagtgcaaaggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatgtcttttatatccttcactaccatgaaaagactagctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcggccatgggagggcgcgccagagatatcagacggaccgactagtgaggcgaactctggataaatgtcttgaataagacgttgaagtatggtttggcgccgaagtcatggacagactacctccaccttacggttgggggcacgttgtcaaatgcgggcgtcagcgggcagacattccggcatggtccacagatcagcaatgtgaacgaattggagattgtgactggaagaggtgatattgtcacttgctcaccagaacagaactctgatctcttccgtgctgctcttggtggtctgggtcagtttgagctccctaggaaggtatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtggagctcaaactgacccagaccaccaagagcagcacggaagagatcagagttctgttctggtgagcaagtgacaatatcacctcttccagtcacaatctccaattcgttcacattgctgatctgtggaccatgccggaatgtctgcccgctgacgcccgcatttgacaacgtgcccccaaccgtaaggtggaggtagtctgtccatgacttcggcgccaaaccatacttcaacgtcttattcaagacatttatccagagttcgcctcactagtgggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttc

SEQ ID NO: 12 - DNA, 260 bp, artificial sequence
  sequence of the inserted HvCKX2 gene fragment, sense orientation
gaggcgaactctggataaatgtcttgaataagacgttgaagtatggtttggcgccgaagtcatggacagactacctccaccttacggttgggggcacgttgtcaaatgcgggcgtcagcgggcagacattccggcatggtccacagatcagcaatgtgaacgaattggagattgtgactggaagaggtgatattgtcacttgctcaccagaacagaactctgatctcttccgtgctgctcttggtggtctgggtcagttt

SEQ ID NO: 13 - DNA, 8906 bp, artificial sequence
  T-DNA region of the vector pMCG/TaCKX1
aattctatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagtgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcactagagtcgaccctagtgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatctagggcatgcgggggttaattaacccccgaattccaatcccaccaaaacctgaacctagcagttcagttgctcctctcagagacgaatcgggtattcaacaccctcataccaactactacgtcgtgtataacggacctcatgccggtatatacgatgactggggttgtacaaaggcagcaacaaacggtgttcccggagttgcgcataagaagtttgccactattacagaggcaagagcagcagctgacgcgtatacaacaagtcagcaaacagataggttgaacttcatccccaaaggagaagctcaactcaagcccaagagctttgcgaaggccctaacaagcccaccaaagcaaaaagcccactgctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcccccatttaaatcccccctcgaggatcaagtgcaaaggtccgccttgtttctcctctgtctcttgatctgactaatcttggtttatgattcgttgagtaattttggggaaagctagcttcgtccacagtttttttttcgatgaacagtgccgcagtggcgctgatcttgtatgctatcctgcaatcgtggtgaacttatgtcttttatatccttcactaccatgaaaagactagctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcggccatgggagggcgcgccagagatatcagacggaccgggccgccgggtgaggaactcgggcgggttcttcaccgacgccgacgtcgccaggatcgtggccgtcgccgcggcgaggaacgctaccaccgtgtacgtcatcgagacgacgctcaactacgacagcgccacggccgcgtccgtggaccaggagctcagctcggtgctggcgacgctgaggcacgaggaggggcacgcgttcgtgcgggacgcgtcgtacctggagttcctggaccgggtgcacggcgaggagggggcgctggacaagatcgggctgtggcgtgtcccgcacccttggctcatcgtgctcgtgccccgctcccgcatcgccgacttcgaccgcggcgtcttcaagggcatcctccagggcaccgacatcgccgggcctctcgtcgtctacccgctcaacaaatccaagtacgcactacgcgtccatcgtgcatgcatgcatgcgatcctgcatggtatgtatcacgcacttaactgacactggctcggtcgtggtttcaggtgggacgacggcatgtcggcggtgacgccagcggaggaggtgttctacgcggtgtcgctgcccttctcgtcggtggccaacgacctgaagcggctggaggcgcagaaccagaagatactgcggttctgcgacctcgccggggtagggtacaaggagtacctggcgcactacacggcccacggcgactgggtccggcacttcggcggcaagtggcagcgcttcgtggagatgaaggacaagtacgaccccaagaggctgctctccccaggccaggacatccctaggaaggtatacatatatgtttataattctttgtttcccctcttattcagatcgatcacatgcatctttcattgctcgtttttccttacaagtagtctcatacatgctaatttctgtaaggtgttgggctggaaattaattaattaattaattgacttgccaagatccatatatatgtcctgatattaaatcttcgttcgttatgtttggttaggctgatcaatgttattctagagtctagagaaacacacccaggggttttccaactagctccacaagatggtgggctagctgacctagatttgaagtctcactccttataattattttatattagatcattttctaatattcgtgtctttttttattctagagtctagatcttgtgttcaactctcgttaaatcatgtctctcgccactggagaaacagatcaggagggtttattttgggtataggtcaaagctaagattgaaattcacaaatagtaaaatcagaatccaaccaattttagtagccgagttggtcaaaggaaaatgtatatagctagatttattgttttggcaaaaaaaaatctgaatatgcaaaatacttgtatatctttgtattaagaagatgaaaataagtagcagaaaattaaaaaatggattatatttcctgggctaaaagaattgttgatttggcacaattaaattcagtgtcaaggttttgtgcaagaattcagtgtgaaggaatagattctcttcaaaacaatttaatcattcatctgatctgctcaaagctctgtgcatctccgggtgcaacggccaggatatttattgtgcagtaaaaaaatgtcatatcccctagccacccaagaaactgctccttaagtccttataagcacatatggcattgtaatatatatgtttgagttttagcgacaatttttttaaaaacttttggtcctttttatgaacgttttaagtttcactgtctttttttttcgaattttaaatgtagcttcaaattctaatccccaatccaaattgtaataaacttcaattctcctaattaacatcttaattcatttatttgaaaaccagttcaaattcttttaggctcaccaaaccttaaacaattcaattcagtggagctcagagcgatcgcagacccggggatgtcctggcctggggagagcagcctcttggggtcgtacttgtccttcatctccacgaagcgctgccacttgccgccgaagtgccggacccagtcgccgtgggccgtgtagtgcgccaggtactccttgtaccctaccccggcgaggtcgcagaaccgcagtatcttctggttctgcgcctccagccgcttcaggtcgttggccaccgacgagaagggcagcgacaccgcgtagaacacctcctccgctggcgtcaccgccgacatgccgtcgtcccacctgaaaccacgaccgagccagtgtcagttaagtgcgtgatacataccatgcaggatcgcatgcatgcatgcacgatggacgcgtagtgcgtacttggatttgttgagcgggtagacgacgagaggcccggcgatgtcggtgccctggaggatgcccttgaagacgccgcggtcgaagtcggcgatgcgggagcggggcacgagcacgatgagccaagggtgcgggacacgccacagcccgatcttgtccagcgccccctcctcgccgtgcacccggtccaggaactccaggtacgacgcgtcccgcacgaacgcgtgcccctcctcgtgcctcagcgtcgccagcaccgagctgagctcctggtccacggacgcggccgtggcgctgtcgtagttgagcgtcgtctcgatgacgtacacggtggtagcgttcctcgccgcggcgacggccacgatcctggcgacgtcggcgtcggtgaagaacccgcccgagttcctcacccggcggccactagtgggtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacc

SEQ ID NO: 14 - DNA, 741 bp, artificial sequence
  sequence of the inserted TaCKX1 gene fragment, sense orientation
tgaggaactcgggcgggttcttcaccgacgccgacgtcgccaggatcgtggccgtcgccgcggcgaggaacgctaccaccgtgtacgtcatcgagacgacgctcaactacgacagcgccacggccgcgtccgtggaccaggagctcagctcggtgctggcgacgctgaggcacgaggaggggcacgcgttcgtgcgggacgcgtcgtacctggagttcctggaccgggtgcacggcgaggagggggcgctggacaagatcgggctgtggcgtgtcccgcacccttggctcatcgtgctcgtgccccgctcccgcatcgccgacttcgaccgcggcgtcttcaagggcatcctccagggcaccgacatcgccgggcctctcgtcgtctacccgctcaacaaatccaagtacgcactacgcgtccatcgtgcatgcatgcatgcgatcctgcatggtatgtatcacgcacttaactgacactggctcggtcgtggtttcaggtgggacgacggcatgtcggcggtgacgccagcggaggaggtgttctacgcggtgtcgctgcccttctcgtcggtggccaacgacctgaagcggctggaggcgcagaaccagaagatactgcggttctgcgacctcgccggggtagggtacaaggagtacctggcgcactacacggcccacggcgactgggtccggcacttcggcggcaagtggcagcgcttcgtggagatgaaggacaagt

SEQ ID NO: 15 - DNA, 36 bp, artificial sequence
  primer CKX2s
ttcggaccgactagtgaggcgaactctggataaatg

SEQ ID NO: 16 - DNA, 36 bp, artificial sequence
  CKX2a
ttcctagggagctcaaactgacccagaccaccaaga

SEQ ID NO: 17 - DNA, 37 bp, artificial sequence
  primer HCV-F
ttcggaccgactagtatccctggctcaacgtgctcgt

SEQ ID NO: 18 - DNA, 37 bp, artificial sequence
  primer HCV-R
ttcctagggagctcagttgaagatgtcttggcccggg

SEQ ID NO: 19 - DNA, 37 bp, artificial sequence
  primer TAC-F
ttcggaccgactagttgaggaactcgggcgggttctt

SEQ ID NO: 20 - DNA, 37 bp, artificial sequence
  primer TAC-R
ttcctaggcccgggacttgtccttcatctccacgaag

SEQ ID NO: 21 - DNA, 20 bp, artificial sequence
  primer qOCS1
cgagcggcgaactaataacg

SEQ ID NO: 22 - DNA, 20 bp, artificial sequence
  primer qOCS2
aattctcggggcagcaagtc

SEQ ID NO: 23 - DNA, 20 bp, artificial sequence
  primer qOCS3
cgagcggcgaactaataacg

SEQ ID NO: 24 - DNA, 20 bp, artificial sequence
  primer qOCS4
aattctcggggcagcaagtc

SEQ ID NO: 25 - DNA, 24 bp, artificial sequence
  primer qOCS5
gccgtccgctctaccgaaagttac

SEQ ID NO: 26 - DNA, 20 bp, artificial sequence
  primer qOCS6
caaaattcgccctggacccg

SEQ ID NO: 27 - DNA, 25 bp, artificial sequence
  primer pM1
tcattcatctgatctgctcaaagct

SEQ ID NO: 28 - DNA, 25 bp, artificial sequence
  primer pM2
tctcgcatatctcattaaagcagga

SEQ ID NO: 29 - DNA, 24 bp, artificial sequence
  primer pM3
atgtccattcgaattttaccgtgt

SEQ ID NO: 30 - DNA, 25 bp, artificial sequence
  primer pM4
gatcagcctaaccaaacataacgaa

SEQ ID NO: 31 - DNA, 22 bp, artificial sequence
  primer pM5
ctcaaagctctgtgcatctccg

SEQ ID NO: 32 - DNA, 21 bp, artificial sequence
  primer pM6
ttattagttcgccgctcggtg

SEQ ID NO: 33 - DNA, 25 bp, artificial sequence
  primer qCKX11
tcgtcgtctacccactcaacaaatc

SEQ ID NO: 34 - DNA, 24 bp, artificial sequence
  primer qCKX12
ttggggtcgtacttgtccttcatc

SEQ ID NO: 35 - DNA, 24 bp, artificial sequence
  primer qCKX21
ggcgaactctggataaatgtcttg

SEQ ID NO: 36 - DNA, 25 bp, artificial sequence
  primer qCKX22
agttctgttctggtgagcaagtgac

(19) United States
(12) Patent Application Publication
(10) Pub. No.: US 2012/0102600 A1
(43) Pub. Date: Apr. 26, 2012

(54) DNA Cassette, Binary Vector, and Strain of A. Tumefaciens and a Method of Producing Cereal Plant of Increased Productivity and/or Root Mass

(76) Inventors: Anna Nadolska-Orczyk, Radzikow (PL); Petr Galuszka, Olomouc (CZ); Wojciech Zalewski, Bialystok (PL); Waclaw Orczyk, Blonie (PL)

(21) Appl. No.: 13/322,299

(22) PCT Filed: May 27, 2010
(86) PCT No.: PCT/PL2010/050019

§ 371 Date: Dec. 19, 2011

(30) Foreign Application Priority Data
May 27, 2009 (PL) 388118

(51) Int. Cl. C12N 15/82; C12N 1/20

(52) U.S. Cl. 800/294; 435/320.1; 435/252.3; 800/278

(57) ABSTRACT

We disclose a DNA cassette and a method of increasing the productivity of cereal plants and/or the root mass of these plants.

The subject of the present invention is a DNA cassette, a binary vector, a strain of A. tumefaciens and a method of producing a serial plant of increased productivity and/or root mass.

In the solution disclosed in international patent application WO02005EP06620 (Schmulling T., Werner T.) the authors achieved an increase in the productivity of plant seeds through the expression of cytokine oxidase in the aleuron layer and/or in the seed embryo as well as demonstrating expression vectors containing nucleic acids encoding cytokinin oxidase of Arabidopsis thaliana under the control of a tissue specific promoter warranting expression in the aleuron and/or the seed embryo. In the solutions described in the published patent applications WO03/050287 and US2005/004-4594, the same authors show a method of stimulating the growth and/or enlargement of the formation of side roots or sucker shoots through the expression of cytokine oxidase or another proteins, reducing the level of active cytokine genes in plants or their parts. Furthermore, they deliver a method of increasing seed size and/or mass, embryo size and/or mass through the expression of cytokinin oxidase or another protein which reduces the levels of active cytokine genes in all plants or in their parts. The goal of the present invention is to deliver a method and tools for its embodiment which facilitate the increased productivity of cereal plants through increasing the number and mass of seeds and/or increasing root mass.

Unexpectedly, the above stated goal was attained using the solution according to the present invention.

The subject of the present invention is a DNA cassette for increasing the productivity of cereal plants and/or a root mass consisting of the following elements: a promoter of expression, a DNA fragment from a coding or non-coding region of the cereal cytokinin oxidase gene CKX in antisense orientation, as well as a 3′ transcription terminator, wherein the cereal cytokinin oxidase gene is selected from among genes expressed in the developing head and/or root, preferentially from a group encompassing the following genes: HvCKX, TaCKX, ZmCKX, ScCKX and AsCKX.

A cassette according to the present invention is preferentially characterized in that the expression of CKX jeans is decreased and its expression leads to the formation of hpRNA, and then siRNA for silencing the expression of at least one of the CKX genes.

The term expression promoter according to the present invention can encompass any promoter active in cereal cells. In particular, this can be a constitutive or induced promoter, or a tissue- or development-specific promoter. According to the present invention, the “fragment of the cereal CKX cytokine oxidase gene” can comprise a sequence of at least 21 nucleotides being a fragment of a coding and/or non-coding sequence of the selected CKX gene whose expression occurs in developing heads and/or roots. In particular, this concerns the example gene HvCKX1, whose expression occurs in young roots, inflorescences and developing heads/grain (FIG. 8) and TaC KX1, whose expression has been observed in grain and drought-stressed seedlings (Galuszka et al. 2004). This also relates to homologues and homelogues of genes of the CKX family in maize, Zea mays L., or ZmCKX text, rye, L., or Secale cerea L., ScCKX or oats, of Avena sativa L., or AsCKX which are expressed in developing heads and/or roots.

In particular, the 3′UTR of the above-mentioned CKX genes can also be the silencing signal. Preferentially, a cassette according to the present invention contains fragments of the cytokine oxidase gene CKX which possesses a nucleotide sequence shown as Sequence 1, or sequence 3 or Sequence 4 or Sequence 5 or a fragment thereof in a sense or anti-sense orientation. Furthermore, these may be coding sequences or fragments of cereal CKX genes available from the NCBI database under the numbers CA 031729, CA 705202, DQ903062, DQ 235927, DQ 238832, CA 603337, DJ 316444 and BJ 322935.

Preferentially, a silencing cassette according to the present invention contains a nucleotide sequence shown as Sequence 6 or Sequence 8. This can also be a silencing cassette containing a fragment of another cereal CKX gene which is expressed in developing heads and/or roots, in a sense and anti-sense orientation as shown in FIG. 1.

The next subject of the present invention is a binary vector containing the DNA cassette defined above.

The next subject of the present invention is a strain of A. tumefaciens containing the vector defined above.

The next subject of the present invention is a method of obtaining a cereal of planned increased productivity characterized in that:

A) A DNA cassette according to the present invention, defined above, is produced,
B) the DNA cassette produced is introduced into the genome of a cereal plant, wherein it is placed under the control of the promoter active in that cell,
C) a cereal plant is derived from the cell thus produced which exhibits increased productivity (increased seed number and/or mass),
D) a cereal plant is derived from a cell thus produced which has an increased root mass.

Preferentially, during stage B), the transformation of the cereal plant cell is performed using a strain of A. tumefaciens containing a binary vector according to the present invention as defined above.

Unexpectedly, it turned out that a significant increase in productivity and root mass of cereal plants was caused by the depression of the expression of the HvCKX1 gene and the inhibition of the activity of the CKX enzyme in these plants, which can be obtained via the introduction into their genome of an expression cassette encoding hpRNAi which is used to silence the gene encoding the enzyme CKX in cereal. Contrary to the suggestions stemming from the prior art cited at the beginning, and according to the present invention the increase of the productivity and root mass of cereal crops was unexpectedly achieved through a totally contrary procedure: the silencing of the expression of particular genes from the CKX family in barley and wheat. The increased productivity (seed mass and number) is positively correlated with root mass. This effect was achieved through the use of a constitutive promoter warranting the expression of the silencing cassette throughout the plant. The expression of the disclosed cassettes/cassette silenced the expression of particular cereal CKX gene/genes, and by the same token reduced the cytokine gene oxidase enzyme expression which led to the increase (and not the reduction) of the level of active cytokine genes in plants and/or their fragments.

### FIGURES

The present description has been supplemented with the attached figures.

FIG. 1. Schematic representation of the silencing cassette containing fragments of the selected cereal CKX gene in a sense and anti-sense orientation.

FIG. 2A. Cloning of the vector pMCG161-HvCKX1. A pal restriction sites in the cloned pMCG161 vector containing the first insert of a fragment of the HvCKX1 gene.

FIG. 2B. Cloning of the vector pMCG161-HvCKX1. Restriction analysis of the pMCG161 vector containing the second insert of the HvCKX1 gene fragment using the enzyme ApaI.

FIG. 3A. Cloning of the vector pMCG161-HvCKX1. Restriction analysis of the vector pMCG161 contain the first insert of the HvCKX2 gene fragment using the enzyme DheI.

FIG. 3B. Map of the vector pMCG161-HvCKX2. Restriction analysis of the vector pMCG161 containing both inserts of the HvCKX2 gene fragment using the enzyme Ehe1.

FIG. 4A. Cloning of the vector pMCG161-TaCKX1. Restriction analysis of the vector pMCG1 61 containing the first insert of the TaCKX1 gene fragment using the enzyme SEC one.

FIG. 4B. Cloning of the vector pMCG161-TaCKX1. Restriction analysis of the vector pMCG1 61 containing both inserts of the TaCKX1 one gene fragment using the enzyme SEC I.

FIG. 5. Structure of the vector pMCG/HvCKX1.

FIG. 6. Structure of the vector pMCG/HvCKX2.

FIG. 7. Structure of the vector pMCG/TaCKX1.

FIG. 8. Relative activity of the enzyme cytokine oxidase/dehydrogenase (CKX) in the roots of T₁ saplings.

FIG. 9A, B, and C. The results of a semi quantitative analysis of the expression of the gene HvCKX1 in various tissues of the strain Scarlett (8) and golden promise (B) as well as HVAC KX two in Golden Promise (C). The upper portion of the gel represents the amplification of the cDNA of the reference gene, actin (qAct) and HvCKX is shown in the lower part (qCKX1, qCKX2). The consecutive lanes show the amplification of cDNA from: 1) 1-day seedlings about 1 cm long, 2) roots from 4 and 5 day seedlings, 3) meristem of 4 and 5 day seedlings, 4) the leaf of the 4 or 5 day seedling, 5) developing leaf of a 2-3 week plant, 6) a developed leaf of a 2-3 week plant, 7) a stem (along with a hypocotyl) from a 6 week plant, 8) early stage inflorescence (3-4 cm long), 9) older inflorescence (6-8 cm long), 10) head during pollination, 11) head a week following pollination, 12) head 2 weeks following pollination.

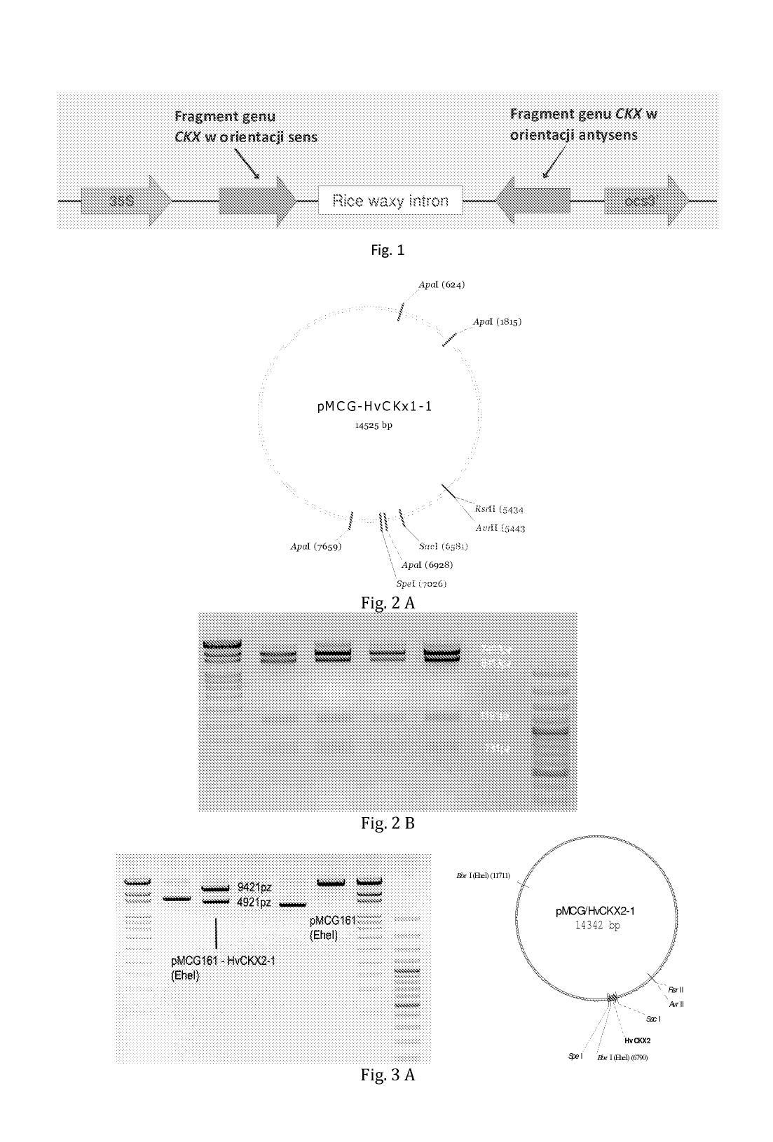

### EXAMPLE 1

### Production of hpRNAi Vectors for Silencing HvCKX1, HvCKX2 and ToCKXl in Cereal Plants

To construct hpRNAi vectors we used the vector pMCG161 (http://www.chromdb.org/mcg161ohtml) containing a silencing cassette with cloning sites for the gene silencing fragment in a sense and anti-sense orientation. The cassettes were prepared based on the sequences of the genes HvCKXl (NCBI accession AF362472) and HvCKX2 (NCBI accession AF540382) of barley as well as ToCKXl (NCBI accession AF362471) of wheat (Galuszka et al., Eur. J. Biochem., 271: 3990-4002). These cassettes were composed of the following functional fragment: a CaMV 35S promoter (others may be used as well, particularly tissue-specific ones), fragment silencing gene in a sense orientation, intron Adh1, a fragment of the silencing gene in an antisense orientation as well as the OCS3′ transcription terminator. The sequence of the silencing cassette containing the fragment of the HvCKX1 gene is shown as Sequence 1. The sequence of the silencing cassette containing the fragment of the HvCKX2 gene is shown as Sequence 2. The sequence of the silencing cassette containing the fragment of the ToCKXl gene is shown as Sequence 3. Following the transformation with a vector containing these cassettes they are integrated with the plant genome, and the siRNA they express regulates expression, by silencing the expression of the above genes.

Following cloning, the vectors were electroporated into E. coli (strain DH5a), isolated and analysed via restriction analysis using several enzymes. Vectors containing cloning constructs were then electroporated into A. tumefaciens, strain Agl1 and again tested using restriction analysis. A detailed description of the cloning to the vector and restriction analysis is shown below.

### Cloning

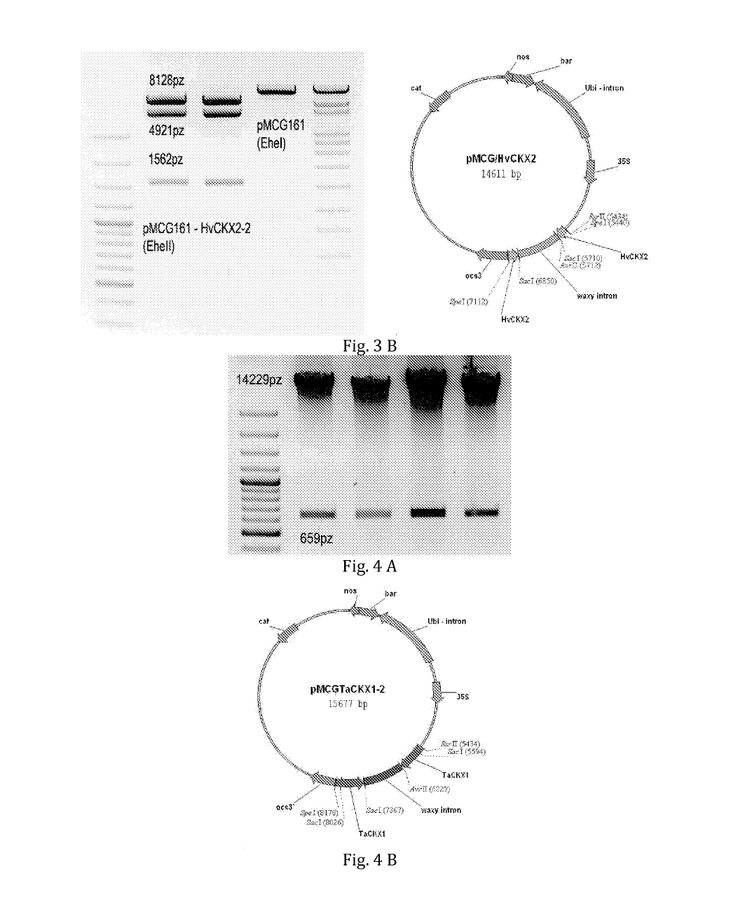

Preparation of pMCG161-HvCKX1. The stages of preparing the vector encompassed:
Amplification of a fragment of HvCKX1 (443 by using primers with the sequences of restriction sites for Sac1, Spe1 as well as Rsrl1 and Avrl1. Amplification on vector pCRT1/NT-TOPO-HvCKX1.
Restriction analysis with the restrictase Apa1.
Purification of the amplified fragment using the Gene Clean KitII.
Digestion of the vector pMCG161 and purified HvCKX1 fragment the restrictase Spe1 (Bcu1).
Purification of the vector and HvCKX1 fragment with the Gene Clean Kit II.
Digestion of the vector and fragment gene the restrictase Sad.
Purification of the digested vector and HvCKX1 fragment with the GeneClean KitII
Ligation of vector pMCG161 with the HvCKX1 fragment (the vector:insert the molar ratio is 1:2).
Purification of the ligation mixture with the GeneClean KitII
Electroporation into E. coli (DH5a)
Inoculation onto plates with an antibiotic (chloramphenicol—35 mg/ml).
Reductive inoculation of selected colonies.
Selected colonies were tested for the presence of the insert HvCKX1, using enzymatic digestion with ApaI. A scheme of the vector with the first cloned fragment of HvCKX1 and ApaI restriction sites is shown in FIG. 2 A).
After confirming the integration of the insert and vector pMCG161, we isolated the plasmid from a selected, positive colony.
Digestion of the vector and insert as well as the HvCKX1 fragment with the restrictases AvrII and Rsrl1
Purification of the digested fragments.
Ligation of the vector with a second HvCKX1 fragment-vector: insert molar ratio is 1:2.
Purification of the ligation mixture.
Electroporation of the purified vector into E. coli (DH5a).
Inoculation of bacteria onto selective medium (chloramphenicol 35 mg/ml)
Reductive inoculation of selected colonies.
Confirmation of the presence of the integration of the second insert in the vector from selected colonies using restriction analysis with the restrictase ApaI. The restriction analysis is shown in FIG. 2 B.
Two-insert vectors were isolated from selected colonies.
Electroporation of the vector pMCG161-HvCKX1 into Agrobacterium tumefaciens (strain Agl1)
Inoculation of bacteria onto MG/L medium with rifampicine (50 mg/l) and chloramphenicol (100 mg/l); reductive inoculation of selected colonies as well as isolation of the plasmid from selected colonies.
Restriction analysis of the vector isolated from Agrobacterium with the enzyme ApaI. Preparation of the vector pMCG161-HvCKX2
Amplification of the fragment of HvCKX2 (289 by using primers with the sequences of restriction sites for Sac1, Spe1 as well as Rsrl1 and Avrl1. Amplification of the vector pDRIVE-HvCKX2.
Confirmation of the amplified fragment with the restrictase Ehe1.
Purification of the amplified fragment using the Gene Clean KitII.
Digestion of the vector pMCG161 and purified HvCKX2 fragment with the restrictases Spe1 (Bcu1) and Sac1.
Purification of the digested vector and HvCKX2 fragment with the GeneClean KitII
Ligation of the vector pMCG161 with the HvCKX2 fragment (vector:insert molar ratio is 1:2)
Purification of the ligation mixture with the GeneClean KitII
Electroporation E. coli (DH5a)
Inoculation of the bacteria onto antibiotic plates (chloramphenicol—35 mg/ml).
Reductive inoculation of selected colonies.
Selected colonies were checked for the presence of the HvCKX2 insert using the restrictase Ehe1(FIG. 3 A).
Following the confirmation of the integration of the insert with the vector pMCG161, we isolated the plasmid from a selected, positive colony.
Digestion of the vector (and insert) as well as the HvCKX2 fragment with the restrictases Avrl1 and Rsrl1
Purification of the digested fragments.
Ligation of the vector with the second HvCKX2 fragment—the vector:insert molar ratio is 1:2.
Purification of the ligation mixture.
Electroporation of the purified vector into E. coli (DH5a).
Inoculation of bacteria onto selective medium (chloramphenicol 35 mg/ml); reductive inoculation of selected colonies
Confirmation of the integration of the second insert into the vector in selected colonies, using restriction analysis with the restrictase Ehe1 (FIG. 3 B).
Two-insert vectors were isolated from selected positive colonies.
Electroporation of the vector pMCG161-HvCKX2 into A. tumefaciens (strain Agl1).
Inoculation onto MG/L medium with rifampicine (50 mg/l) and chloramphenicol (100 mg/l).
Reductive inoculation of selected colonies as well as isolation of the plasmid from selected colonies.
Restriction analysis of the vector isolated from Agrobacterium with the restrictase Ehe1.
Preparation of the vector pMCG161-TaCKX1
Amplification of a fragment of ToCKXl (770 by using primers containing the sequences of restriction sites for Spel and Xmal as well as Rsrll and Avrll. Amplification onto the vector pDRIVE-TaCKXl.
Confirmation of the amplified fragment with the restrictase Sacl.
Purification of the amplified fragment using the Gene Clean KitII.
Digestion of the vector pMCG161 and purified ToCKXl fragment with the restrictases Spe1 (Bcu1) and Xma1.
Purification of the digested vector and ToCKXl fragment with the GeneClean KitII.
Ligation of the vector pMCG161 and the ToCKXl fragment (vector:insert molar ratio is 1:2).
Purification of the ligation mixture using GeneClean KitII.
Electroporation into E. coli (DH5a)
Inoculation onto antibiotic plates (chloramphenicol—35 mg/ml).
Reductive inoculation of selected colonies.
Selected colonies checked for the presence of the TaCKX1 insert using digestion with the SacI restrictase (FIG. 4 A).
After checking the integration of the insert into the vector pMCG161, we isolated the plasmid with the insert from a selected, positive colony.
Digestion vector (with insert) as well as ToCKX1 fragment with restrictases Avrll and Rsrll.
Purification of the digested fragment.
Ligation of the vector z with the second TaCKX1 fragment-vector:insert molar ratio is 1:2.
Purification of the ligation mixture.
Electroporation of the purified vector into E. coli (DH5a).
Inoculation of the bacteria onto selection medium (chloramphenicol 35 mg/ml); reductive inoculation of selected colonies.
Checking the integration of the second insert into the vector in selected colonies with restriction analysis using the restrictase SacI (FIG. 4 B).
A two-insert vector was isolated from positive colonies.
Electroporation vector pMCG161-ToCKXl to A. tumefaciens (strain Agl1)
Inoculation of bacteria onto MG/L medium with rifampicine (50 mg/l) and chloramphenicol (100 mg/l); reductive inoculation of selected colonies and plasmid isolation from selected colonies.
restriction analysis of the vector isolated from Agrobacterium using the restrictase Sad. A. tumefaciens strains thus produced, containing appropriate binary vectors, were used to the transformation of cereal genes.

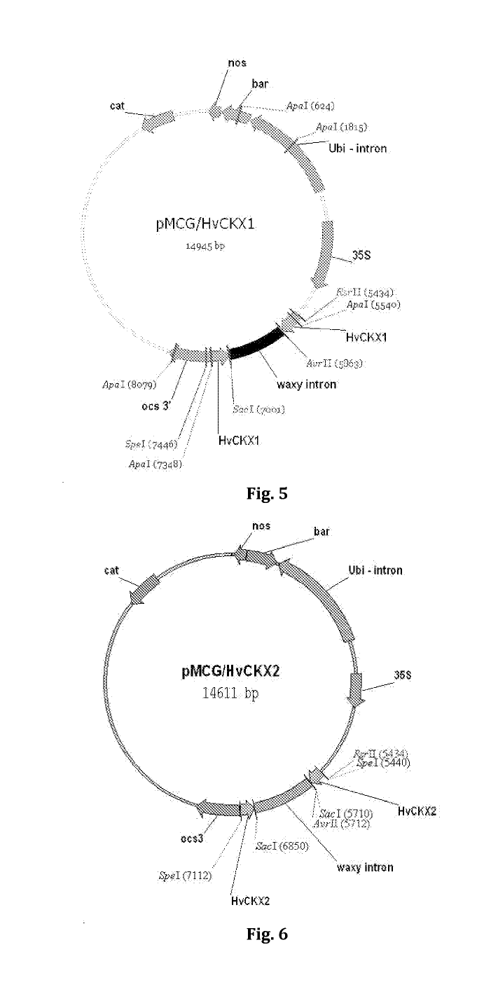

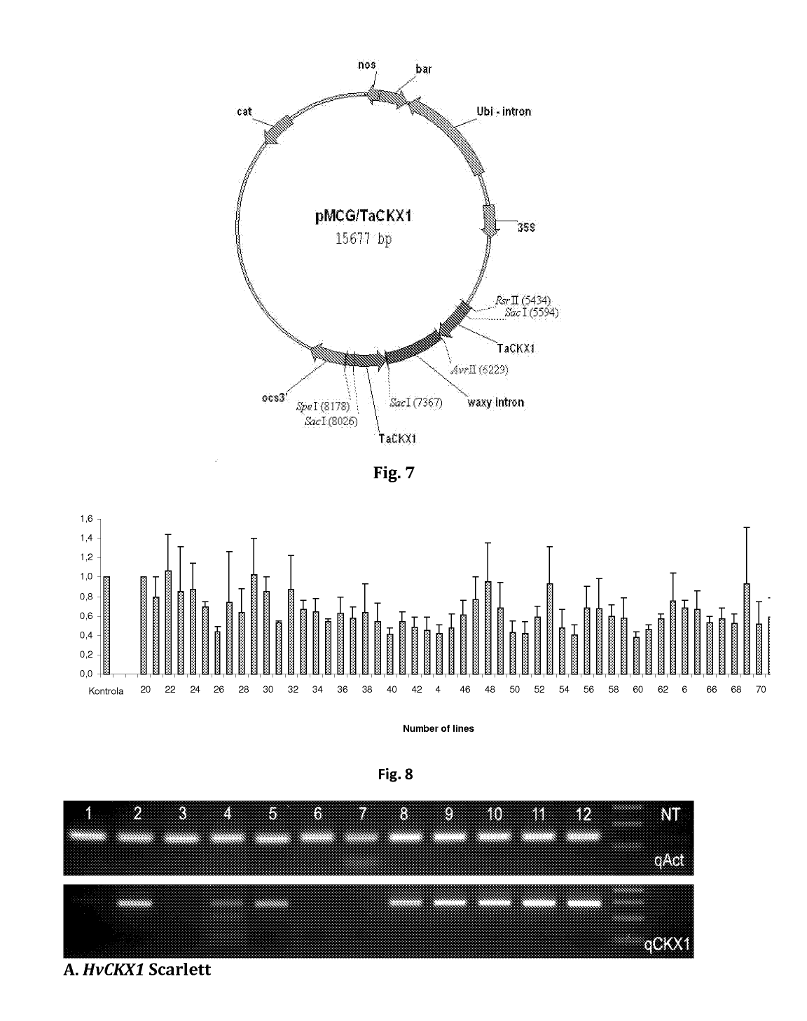

The binary vector pMCG/HvCKX1 containing the following functional elements: T-DNA with a selection cassette as well as a silencing cassette is shown in FIG. 5, the nucleotide sequence of the T-DNA region of this vector is shown as Sequence 9, and the sequence of the inserted fragment of the gene HvCKX1 in a sense orientation as Sequence 10. The binary vector pMCG/HvCKX2 containing the following functional elements: T-DNA with a selection cassette as well as a silencing cassette is shown in FIG. 6, the nucleotide sequence of the T-DNA region of this vector is shown as Sequence 11, and the sequence of the inserted fragment of the gene HvCKX1 in a sense orientation as Sequence 12. The binary vector pMCG/TaCKX1 containing the following functional elements: T-DNA with a selection cassette as well as a silencing cassette is shown in FIG. 7, the nucleotide sequence of the T-DNA region of this vector is shown as Sequence 13, and the sequence of the inserted fragment of the gene ToCKXl in a sense orientation as Sequence 14. To clone the gene fragments into the silencing cassettes, we used primers shown in Table 1.

TABLE 1
Primer sequences designed and used to clone
fragments for silencing genes in a sense and
antisense orientation into a silencing
cassette in the vector pMCG161.

| Primer | Sequence | Use |
|---|---|---|
| CKX2s | TTCGGACCGACTAGTGAGGCGAACTCTG | cloning cassette |
| | GAT AAATG | silencing gene |
| CKX2a | TTCCTAGGGAGCTCAAACTGACCCAGAC | HvCKX2 |
| | CACCAAGA | |
| HCV-F | TTCGGACCGACTAGTATCCCTGGCTCAA | cloning cassette |
| | CGTGCTCGT | silencing gene |
| HCV-R | TTCCTAGGGAGCTCAGTTGAAGATGTCT | HvCKX1 |
| | TGGCCCGGG | |
| TAC-F | TTCGGACCGACTAGTTGAGGAACTCGGG | cloning cassette |
| | CGGGTTCTT | silencing gene |
| TAC-R | TTCCTAGGCCCGGGACTTGTCCTTCATC | TaCKX1 |
| | TCCACGAAG | |

### EXAMPLE 2

### Production of the Transformed Cereal Plants

We transformed two strains of barley, Golden Promise and Scarlett, and wheat (Polish strains Torka and Kontesa) using a RNAi vector (via a gene modification method with the use of Agrobacterium tumefaciens as well as via a biolisitic method).

In vitro culture method and transformation using A. tumefaciens

Extraction of barley and wheat embryos: heads 12-14 days following pollination (wheat) or 8-18 days post pollination (barley) are collected and then the grain is husked and sterilized.

Seed sterilization: rinsing in 70% ethanol for a minute; decant alcohol, add 2-3 drops of Tween 20; immerse in 0.1% HgCl; rinse in sterilizing buffer for 3-4 minutes; rinse with sterile water 3 times, for 5, 10 and 15 minutes respectively; decant water; isolate and seed. Seed 20 embryos on a plate with modified MSB3 medium for barley and MSB6 for wheat embryos.

MSB3 medium composition (modified acc. To Wan and Lemaux, 1994; Trifonova et al. 2001, Przetakiewicz et al. 2003): macro- and microelements acc. to Murashige and Skoog, (1962); 30 g/l maltose, 500 mg/l hydrolysed casein, 1.234 mg/l CuS0₄, 2.5 mg/l DICAMBA, 3.0 g/l GelRite, 0.02 g/l thiamine, 5 g/l myoinositol, 13.8 g/l proline. pH 5.6-5.8 MSB6 medium composition (modified acc. to Przetakiewicz et al. 2003): the base MSB medium has macro and microelements according to Murashige and Skoog, (1962) and vitamins acc. To Gamborg et al. (1968). The medium MSB6 contains the components of MSB as well as: 30 g/l saccharose, 2 mg/l picrolam, 1 mg/12.4-3.0 g/l GelRite; pH 5.6-5.8. The culture is maintained in a culture room at a temperature of 22-24° C., in a 16/8 photoperiod (day/night), 50 μm-²s-¹ illumination under a filter tissue cover for 2-3 days. Preparation of the A. tumefaciens Agl1 strain for transformation—the bacterial culture is initiated sufficiently ahead of time (1-2 days). The culture is maintained in MG/L medium with the appropriate antibiotics (rifampicine 50 mg/l, chloramphenicol—70 mg/l). When the culture reaches an appropriate stage (OD₆₀₀=0.6-1.2), the flask contents are transferred into centrifuge tubes, which are centrifuged for 10 minutes at 6000 rpm, 4° C. After centrifugation, the supernatant level is marked, and it is decanted. Fresh MSB3 medium is poured into the marked level. Acetosyringon is added. The tubes are placed on a shaker in order to dissolve the bacterial precipitate in the medium.

Transformation/inoculation of immature embryos with A. tumefaciens—The prepared bacterial suspension with acetosyringon is dropped onto each embryo. Two plates with untreated embryos are maintained (control). The culture is maintained in a culture room under standard conditions (as above), under lights and a cover of tissue.

Barley embryos were transformed with the A. tumefaciens Agl1 strain containing the vectors pMCG/HvCKX1 and pMCG/HvCKX2. Wheat was transformed with A. tumefaciens Agl1 with the vectors pMCG161/TaCKX1, pMCG/HvCKX1 and pMCG161/HvCKX2.

Passaging—After three days of post-inoculation with the bacteria, the embryos are transferred in groups of 6 onto a selection medium with the appropriate antibiotics (phosphinotricine—2 mg/l, thimentin—150 mg/l). At the same time, a positive control of the regeneration was maintained: untransformed embryos on non-antibiotic medium as well as a negative control: untransformed embryos on antibiotic medium. After four weeks we transferred embryos/callus lines in groups of 4 onto R2-MSB medium (Przetakiewicz et al. 2003) containing 1 mg/l BA and 0.2 mg/l IAA with antibiotics as above. After another 2-4 weeks, the regenerating plants are transferred onto R2-MSB medium with antibiotics for further growth. The growing plants (over 1 cm) are transferred into 0.5 l jars and into 1/2 MS medium (half micro- and macroelement concentration acc. to Murashige and Skoog, 1962) with antibiotics. Successfully growing and rooting plants are planted into pots with fresh soil, and freshly planted plants are left for several days under cover to adapt them to the new conditions.

Material for analysis can be collected from the growing plants.

We obtained 108 potentially transgenic plants from 75 callus lines. The results are shown in Table 2.

TABLE 2
Numbers of transformation explants (immature embryos), selected plants
as well as lines and transformation efficiency in the individual
experiments using the silencing, control (pMCG161) and expression vector
and via the Agrobacterium and biolistic methods.

| | | | | | |
|---|---|---|---|---|---|
| | No. Exp./ | Num- | Number of | num- | Trans- |
| | vector | ber | plants selected | ber | formation |
| | silencing | ex- | following the | of | efficiency |
| strain | and control | plants | transformation | lines | (%) |

Transformation using A. Tumefaciens

| | | | | | |
|---|---|---|---|---|---|
| Golden | 1. pMCG/CKX1 | 825 | 52 | 32 | 6.3 |
| Promise | 4. pMCG/CKX2 | 421 | 36 | 28 | 8.6 |
| | 4. pMCG161 | 100 | 4 | 3 | 4.0 |
| | 5. pMCG/CKX2 | 75 | 5 | 4 | 6.7 |
| | 6. pMCG/CKX2 | 440 | 1 | 1 | 0.2 |
| | 6. pMCG161 | 75 | 0 | 0 | 0 |
| | 7. pMCG/CKX2 | 231 | 0 | 0 | 0 |
| | total | 2167 | 98 | 68 | 4.52 |
| Scarlett | 1. pMCG/CKX1 | 633 | 1 | 1 | 0.16 |
| | 4. pMCG/CKX2 | 507 | 1 | 1 | 0.20 |
| | 6. pMCG/CKX2 | 237 | 0 | 0 | 0 |
| | 6. pMCG161 | 125 | 0 | 0 | 0 |
| | 7. pMCG/CKX2 | 335 | 0 | 0 | 0 |
| | total | 1837 | 2 | 2 | 0.11 |
| Kontesa | 4. pMCG/CKX2 | 715 | 0 | 0 | 0 |
| | 4. pMCG161 | 100 | 0 | 0 | 0 |
| | 5. pMCG/TaCKX1 | 76 | 0 | 0 | 0 |
| | 6. pMCG/TaCKX1 | 322 | 0 | 0 | 0 |
| | 7. pMCG/TaCKX1 | 71 | 0 | 0 | 0 |
| | total | 1284 | 0 | 0 | 0 |
| Torka | 4. pMCG/CKX2 | 550 | 0 | 0 | 0 |
| | 4. pMCG161 | 100 | 0 | 0 | 0 |
| | 5. pMCG/TaCKX1 | 260 | 0 | 0 | 0 |
| | 6. pMCG/TaCKX1 | 344 | 0 | 0 | 0 |
| | total | 1254 | 0 | 0 | 0 |
| Wanad | 4. pMCG/CKX2 | 1000 | 0 | 0 | 0 |
| | 4. pMCG161 | 125 | 0 | 0 | 0 |
| | 5. pMCG/TaCKX1 | 350 | 0 | 0 | 0 |
| | total | 1475 | 0 | 0 | 0 |

Biolistic transformation

| | | | | | |
|---|---|---|---|---|---|
| Golden | 2. HvCKX2linear | 251 | 5 | 4 | 1.99 |
| Promise | | | | | |
| Scarlett | 2. HvCKX2linear | 620 | 3 | 1 | 0.48 |

Phenotypic analysis of T₀ plants (genetically modified plants regenerated in vitro), production and analysis of T₁ progeny lines (from each T₀ one a line in which characteristics are inherited).

Seeds were obtained from all plants regenerated and selected on selection media. These were counted and weighed, and the mass per thousand seeds was calculated. The number of seeds ranged from 36 to 332 pieces, and the thousand seed mass (TSM) was from 12 to 41.28 g.

### Genetic Analysis

Using genetic analyses (mainly PCR), we confirmed that the resulting T₀ are transgenic. For this purpose, we designed and used 7 pairs of specific primers, whose sequences are shown in Table 3.

TABLE 3
Specific primers designed for the analysis
of potentially transgenic plants selected
after the following transformation.

| Primer | Sequence | use |
|---|---|---|
| qOCS1 | CGAGCGGCGAACTAATAACG | qPCR (quantitative |
| qOCS2 | AATTCTCGGGGCAGCAAGTC | PCR) for the |
| | | silencing |
| | | cassette |
| qOCS3 | CGAGCGGCGAACTAATAACG | qPCR of the |
| qOCS4 | AATTCTCGGGGCAGCAAGTC | silencing |
| | | cassette |
| qOCS5 | GCCGTCCGCTCTACCGAAAGTTAC | qPCR of the |
| qOCS6 | CAAAATTCGCCCTGGACCCG | silencing |
| | | cassette |
| pM1 | TCATTCATCTGATCTGCTCAAAGCT | PCR of the |
| pM2 | TCTCGCATATCTCATTAAAGCAGGA | silencing |
| | | cassette |
| pM3 | ATGTCCATTCGAATTTTACCGTGT | PCR of the |
| pM4 | GATCAGCCTAACCAAACATAACGAA | silencing |
| | | cassette |
| pM5 | CTCAAAGCTCTGTGCATCTCCG | PCR of the |
| pM6 | TTATTAGTTCGCCGCTCGGTG | silencing |
| | | cassette |

### EXAMPLE 3

### Silencing of the HvCKXl Gene Using hpRNA/siRNA Leads to an Increased Productivity and Root Mass in Cereal Plants

We analysed the gene silencing effect on HvCKXl as well as the phenotypic characteristics in 52 genetically modified lines of Golden Promise and 2 Scarlett lines.

The first stage of analyzing T₁ plants was to measure the activity level of the cytokinin oxidase/dehydrogenase enzyme (CKX) in the roots of plants resulting from transformation with a silencing vector for the gene HvCKX1. For this purpose, we sprouted groups of 5 of each T0 plant, cut off the root at the base, weighed them individually and pooled the roots from five plants for the measurements. The experiment was performed thrice (for 3×5 T₁ plants per line). The results of the relative activity of cytokinin oxidase/dehydrogenase (CKX) are shown with standard deviations for 52 analysed T₁ lines are shown in FIG. 8.

The relative values of these measurements, assuming the control measurement as 1.00 (line regenerated in vitro, not transformed) varied from 0.38 to 1.23. A significantly lower cytokinin oxidase/dehydrogenase activity level was noted in 40 lines. In order to compare the enzymatic activity with line productivity as well as root mass, they were divided into three groups: 1) with a relative CKX activity level below 0.59, 2) with a relative activity level from 0.6 to 0.79 and 3) above 0.8. The first two groups, encompassing 40 lines, exhibited a significantly lower enzymatic activity level in relation to the third group, which was similar to the control. A compilation of the results, encompassing seed number, thousand seed mass in T₀ plants as well as an average root mass (of the 15 progeny plants, T₁) and relative CKX activity in the roots is shown in Table 4.

TABLE 4
Three groups of lines: with CKX enzymatic activity
below 0.59; from 0.6 to 0.79 and above 0.8 and their
corresponding productivity and root mass levels.

| | | | | | | |
|---|---|---|---|---|---|---|
| | | | | thou- | average | |
| | | num- | | sand | root | |
| | | ber | seed | seed | mass | relative |
| Line | T₀ | of | mass | mass | (mg) | CKX |
| No. | plant | seeds | (mg) | (g) | in T₁ | activity |

Enzymatic activity <0.59

| | | | | | | |
|---|---|---|---|---|---|---|
| 25 | 2G/4 | 210 | 8522 | 40.58 | 40.21 | 0.44 ± 0.05 |
| 30 | 5G/2B | 63 | 2053 | 32.59 | 26.50 | 0.53 ± 0.02 |
| 34 | 5G/4 | 214 | 7567 | 35.36 | 27.95 | 0.54 ± 0.03 |
| 36 | 5G/5B | 185 | 6223 | 33.64 | 33.85 | 0.58 ± 0.11 |
| 38 | 5G17A | 203 | 6382 | 31.44 | 31.92 | 0.54 ± 0.19 |
| 39 | 5G/7B | 197 | 6467 | 32.83 | 30.92 | 0.41 ± 0.06 |
| 40 | 5G/8 | 217 | 6564 | 30.25 | 29.29 | 0.54 ± 0.1 |
| 41 | 5G/9 | 99 | 3221 | 32.54 | 31.33 | 0.49 ± 0.1 |
| 42 | 5G/10A | 184 | 4711 | 25.60 | 29.63 | 0.45 ± 0.14 |
| 43 | 5G/10B | 239 | 7850 | 32.85 | 33.90 | 0.42 ± 0.08 |
| 44 | 5G/11 | 157 | 5031 | 32.04 | 37.80 | 0.48 ± 0.15 |
| 49 | 5G/14A | 233 | 8470 | 36.35 | 33.00 | 0.43 ± 0.12 |
| 50 | 5G/14B | 217 | 6725 | 30.99 | 32.92 | 0.43 ± 0.12 |
| 51 | 5G/15A | 55 | 1381 | 25.11 | 34.13 | 0.59 ± 0.11 |
| 53 | 5G/16B | 120 | 4091 | 34.09 | 29.13 | 0.48 ± 0.19 |
| 54 | 5G/17 | 142 | 3875 | 27.29 | 21.45 | 0.41 ± 0.11 |
| 57 | 5G/19/C | 71 | 2305 | 32.46 | 31.88 | 0.59 ± 0.12 |
| 58 | 5G/20A | 126 | 4061 | 32.23 | 37.40 | 0.58 ± 0.20 |
| 59 | 5G/20B | 217 | 6685 | 30.81 | 23.58 | 0.38 ± 0.06 |
| 60 | 5G/20C | 174 | 5743 | 33.01 | 27.79 | 0.46 ± 0.04 |
| 61 | 5G/20D | 177 | 6230 | 35.20 | 28.10 | 0.57 ± 0.05 |
| 65 | 5G/23B | 170 | 7018 | 41.28 | 27.83 | 0.53 ± 0.07 |
| 66 | 5G/24 | 142 | 4796 | 33.77 | 30.90 | 0.58 ± 0.11 |
| 67 | 5G/25 | 185 | 6080 | 32.86 | 30.25 | 0.53 ± 0.09 |
| 69 | 6G/1A | 143 | 3721 | 26.02 | 37.00 | 0.52 ± 0.23 |
| 70 | 6G11B | 235 | 7939 | 33.78 | 33.25 | 0.59 ± 0.20 |
| average | 26 | 168.27 | 5527.35 | 32.50 | 31.23 | 0.50 ± 0.11 |

enzymatic activity 0.6-0.79

| | | | | | | |
|---|---|---|---|---|---|---|
| 24 | 2G/3C | 90 | 2175 | 24.17 | 16.13 | 0.69 ± 0.06 |
| 27 | 5G/1B | 94 | 2542 | 27.04 | 18.50 | 0.63 ± 0.25 |
| 32 | 5G/3A | 86 | 3330 | 38.72 | 29.38 | 0.67 ± 0.09 |
| 33 | 5G/3B | 265 | 8678 | 32.75 | 30.13 | 0.65 ± 0.14 |
| 35 | 5G/5A | 260 | 6993 | 26.90 | 25.33 | 0.63 ± 0.17 |
| 37 | 5G/6 | 195 | 7477 | 38.34 | 30.88 | 0.64 ± 0.29 |
| 45 | 5G/12A | 49 | 1476 | 30.12 | 32.20 | 0.62 ± 0.15 |
| 46 | 5G/12A-1 | 166 | 5509 | 33.19 | 44.50 | 0.77 ± 0.23 |
| 48 | 5G/13 | 134 | 3847 | 28.71 | 30.25 | 0.68 ± 0.27 |
| 55 | 5G/18 | 202 | 7016 | 34.73 | 34.83 | 0.69 ± 0.22 |
| 56 | 5G/19A | 177 | 6181 | 34.92 | 38.08 | 0.68 ± 0.31 |
| 62 | 5G/21 | 206 | 7532 | 36.56 | 25.13 | 0.76 ± 0.28 |
| 63 | 5G/22 | 89 | 2608 | 29.30 | 24.05 | 0.68 ± 0.08 |
| 64 | 5G/23A | 148 | 5030 | 33.99 | 18.50 | 0.67 ± 0.19 |
| average | 14 | 154.36 | 5028.14 | 32.10 | 28.42 | 0.68 ± 0.20 |

enzymatic activity >0.8

| | | | | | | |
|---|---|---|---|---|---|---|
| 19 | KOP 4G/1 | 50 | 489 | 9.78 | data n/a | 1.00 ± 0.00 |
| 20 | 2G/1 | 36 | 432 | 12.00 | 9.83 | 0.80 ± 0.21 |
| 21 | 2G/2 | 66 | 1430 | 21.67 | 16.53 | 1.07 ± 0.37 |
| 22 | 2G/3A | 77 | 1786 | 23.19 | 27.00 | 0.85 ± 0.47 |
| 23 | 2G/3B | 80 | 2056 | 25.70 | 25.42 | 0.87 ± 0.27 |
| 28 | 5G11C | 145 | 3584 | 24.72 | 26.54 | 1.03 ± 0.37 |
| 29 | 5G/1D | 143 | 3116 | 21.79 | 18.00 | 0.85 ± 0.15 |
| 31 | 5G/2B | 214 | 6530 | 30.51 | 24.04 | 0.88 ± 0.35 |
| 47 | 5G/12B | 100 | 3082 | 30.82 | 27.88 | 0.96 ± 0.40 |
| 52 | 5G116A | 177 | 5978 | 33.77 | 27.60 | 0.93 ± 0.38 |
| 68 | 5G/28 | 148 | 4850 | 32.77 | 19.45 | 0.93 ± 0.58 |
| 71 | 6G/2 | 110 | 3976 | 36.15 | 31.42 | 0.85 ± 0.40 |
| average | 12 | 117.82 | 3347.27 | 26.64 | 23.06 | 0.92 ± 0.33 |

A comparison of the averages of three groups shows a clear positive correlation between productivity and root mass in lines with depressed CKX activity. In the first group, encompassing plants with a relative enzymatic activity level below 0.59 (average=0.50±0.11). The average seed number in T₀ plants was 168.27; thousand seed mass was 32.5 g, and the average root mass of T₁ seedlings was 31.23 mg. In the second line, with the average relative activity level of 0.6 to 0.79 (average=0.68±0.20) the values were, respectively: 154.36; 32.10 g and 28.42 mg. In the third group, encompassing lines with activities approaching those of the control (average=0.92±0.33) we obtained on average 117.82 seeds, with a mass of 26.64 g and average root mass in T₁ seedlings of 23.06 mg. The results of decreased enzymatic activity levels in 40 transgenic lines of the Golden Promise strain of barley attest to, on average, a significantly decreased expression of the silenced gene HvCKX1. One cannot also discount the possibility of other genes from this family by the construct used, whose expression occurs in the root and which have sequences homologous to those used in the silencing construct. A consequence of the reduced activity of the cytokinin oxidase/dehydrogenase enzyme is an increase in root mass, and in a portion of the genes, an increase of the line's productivity (Table 4). The lower the CKX activity level, the higher the number of seeds obtained as well as higher thousand seed masses in T₀ plants as well as average root mass in T₁ plants.

The relative, quantitative measurement of the expression of the HvCKXl gene in transgenic T₁ plants of the Golden Promise strain, transformed with a silencing construct for this gene. During the second stage of analysis of T1 plants, we performed measurements of the expression of the HvCKXl gene in the roots of four-day seedlings. We sprouted 6 seeds from each T₀ line, individually cut off the root at the base and weighed it. RNA was isolated from a portion of the root and then transcribed into cDNA.

To perform the quantitative analysis of the expression of the HvCKX2 gene, we designed and used the following primers:

| Primer | Sequence | use |
|---|---|---|
| qCKX11 | TCGTCGTCTACCCACTCAACAAATC | RT-PCR and qRT- |
| qCKX12 | TTGGGGTCGTACTTGTCCTTCATC | PCR of the |
| | | HvCKX1 gene |

the results of these measurements in selected T₁ plants are shown in Table 5.

TABLE 5
relative quantitative measurement of the expression of the gene HvCKX1 in transgenic T₁ generation
plants of the strain Golden Promise transformed with a construct for silencing this gene.

| | | | | | | | rel. |
|---|---|---|---|---|---|---|---|
| | | | | ground | | efficiency | expression |
| | | | root | root | RNA | of isolation | of CKX1 in |
| | | | mass | material | conc. | from root | the root |
| plant identifier | | plant | (mg) | (mg) | (ng/ul) | (ng/mg) | MCt |
| 1 | GP/6 | Golden Prom. | 64 | 31 | 264.39 | 255.86 | 1.00^{a,d,e,f} |
| 2 | GP/7 | Golden Prom. | 56 | 32 | 402.31 | 377.17 | 1.00^{b} |
| 4 | GP in vitro 1/6 | II FG KP/1A | 81 | 43 | 414.2 | 288.98 | 1.11^{a} |
| 5 | GP in vitro 1/7 | II FG KP/1A | 81 | 45 | 465.45 | 310.30 | 1.09^{b} |
| 6 | GP in vitro 1/8 | II FG KP/1A | 56 | 27 | 470.43 | 522.70 | 1.00^{c} |
| 7 | 28/6 | 5G/1C | 44 | 23 | 240.3 | 313.43 | 1.89^{a} |
| 8 | 28/7 | 5G/1C | 42 | 23 | 453.37 | 591.35 | 1.22^{b} |
| 10 | 28/9 | 5G/1C | 43 | 30 | 570.5 | 570.50 | 1.16^{d} |
| 12 | 28/11 | 5G/1C | 64 | 44 | 670.5 | 457.16 | 0.80^{f} |
| 14 | 30/7 | 5G/2B | 71 | 45 | 748.77 | 499.18 | 0.97^{b} |
| 16 | 30/9 | 5G/2B | 46 | 24 | 507.5 | 634.38 | 0.81^{d} |
| 19 | 38/6 | 5G/7A | 55 | 35 | 424.41 | 363.78 | 1.93^{a} |
| 20 | 38/7 | 5G/7A | 52 | 40 | 609.55 | 457.16 | 1.23^{b} |
| 25 | 43/6 | 5G/10B | 34 | 18 | 187.17 | 311.95 | 2.51^{a} |
| 26 | 43/7 | 5G/10B | 69 | 42 | 460.64 | 329.03 | 1.89^{b} |
| 27 | 43/8 | 5G/10B | 73 | 52 | 722.60 | 416.88 | 0.82^{c} |
| 31 | 52/6 | 5G/16A | 69 | 39 | 347.51 | 267.32 | 1.02^{a} |
| 32 | 52/7 | 5G/16A | 66 | 26 | 346.9 | 400.27 | 2.72^{b} |
| 35 | 52/10 | 5G/16A | 60 | 40 | 583.8 | 437.85 | 0.86^{e} |
| 36 | 52/11 | 5G/16A | 60 | 32 | 567.2 | 531.75 | 1.06^{f} |
| 37 | 59/6 | 5G/20B | 71 | 40 | 460.35 | 345.26 | 1.14^{a} |
| 38 | 59/7 | 5G/20B | 56 | 32 | 448.15 | 420.14 | 1.09^{b} |
| 42 | 59/11 | 5G/20B | 58 | 37 | 438.1 | 355.22 | 1.11^{f} |
| 43 | 70/6 | 6G/1B | 49 | 27 | 348.3 | 387.00 | 1.46^{a} |
| 44 | 70/7 | 6G/1B | 62 | 39 | 609.28 | 468.68 | 1.23^{b} |
| 45 | 70/8 | 6G/1B | 47 | 23 | 463.91 | 605.10 | 0.89^{c} |
| average mass per expression level >0.80 | | | 58.81 | | | | |
| 9 | 28/8 | 5G/1C | 44 | 27 | 492.92 | 547.69 | 0.60^{c} |
| 18 | 30/11 | 5G/2B | 102 | 77 | 1009.9 | 393.47 | 0.61^{f} |
| 39 | 59/8 | 5G/20B | 45 | 21 | 638.86 | 912.66 | 0.64^{c} |
| 41 | 59/10 | 5G/20B | 37 | 17 | 525.5 | 927.35 | 0.6^{ge} |
| 28 | 43/9 | 5G/10B | 51 | 19 | 437.3 | 690.47 | O.72^{d} |
| 30 | 43/11 | 5G/10B | 72 | 42 | 577.5 | 412.50 | 0.71^{f} |
| 34 | 52/9 | 5G/16A | 53 | 21 | 443 | 632.86 | 0.73^{d} |
| 48 | 70/11 | 6G/1B | 70 | 36 | 457.9 | 381.58 | 0.73^{f} |
| 17 | 30/10 | 5G/2B | 66 | 44 | 655.1 | 446.66 | 0.76^{e} |
| 24 | 38/11 | 5G/7A | 80 | 54 | 737.1 | 409.50 | 0.79^{f} |
| average mass per expression level 0.60-0.79 | | | 62.00 | | | | |
| 46 | 70/9 | 6G/1B | 44 | 15 | 312.7 | 625.40 | 0.10^{d} |
| 11 | 28/10 | 5G/1C | 82 | 58 | 714.8 | 369.72 | 0.31^{e} |
| 22 | 38/9 | 5G/7A | 85 | 59 | 786.2 | 399.76 | 0.41^{d} |
| 47 | 70/10 | 6G/1B | 71 | 52 | 737.6 | 425.54 | 0.45^{e} |
| 15 | 30/8 | 5G/2B | 57 | 34 | 699.95 | 617.60 | 0.51^{c} |
| 40 | 59/9 | 5G/20B | 42 | 19 | 420.6 | 664.11 | 0.54^{d} |
| 29 | 43/10 | 5G/10B | 80 | 52 | 604.2 | 348.58 | 0.54^{e} |
| 23 | 38/10 | 5G/7A | 68 | 41 | 783.3 | 573.15 | 0.57^{e} |
| 13 | 30/6 | 5G/2B | 99 | 57 | 517.77 | 272.51 | 0.58^{a} |
| 33 | 52/8 | 5G/16A | 42 | 18 | 390.08 | 650.13 | 0.59^{c} |
| 21 | 38/8 | 5G/7A | 58 | 40 | 719.82 | 539.87 | 0.59^{c} |

| | |
|---|---|
| average mass per expression level <0.59 | 66.18 |

The T₁ plants tested in table 6 were segregated into three groups depending on the intensity of the silencing of the expression of the gene in question. Among T₁ plants exhibiting a relative expression level of HvCKX1 in excess of 0.80, the average root mass in a 4-day seedling was 58.81 mg. Among plants with a relative expression from 0.60 to 0.79 the average root mass was 62.00 mg. In the group with the lowest relative expression of HvCKX1 (below 0.59), the average root mass was 66.18 mg.

Conclusions: We demonstrated a positive correlation between productivity and root mass of the examined lines and the lowered expression of the HvCKX1 gene as well as CKX enzymatic activity. In the first group, encompassing plants with a relative enzymatic activity below 0.59 (average=0.50±0.11), the average number of seeds in T₀ plants was 168.27; thousand seed mass was 32.5 g, and the average root mass in T₁ seedlings was 31.23 mg. In the second group, with a relative enzyme activity level from 0.6 to 0.79 (average=0.68±0.20) the values were, respectively, 154.36, 32.10 g and 28.42 mg. In the third group, encompassing lines with an activity level close to that of the control (average=0.92±0.33) we obtained on average 117.82 seeds with a mass of 26.64 g and an average root mass in T₁ seedlings of 23.06 mg. The results of the lowered enzymatic activity in 40 transgenic lines of Golden Promise barley match the results of the lowered expression level of the silenced gene HvCKXl in the examined plants. One cannot also discount the possibility of other genes from this family by the construct used, whose expression occurs in the root and which have sequences homologous to those used in the silencing construct. A consequence of the reduced expression of the HVCKX1 gene is a reduction in the activity of cytokinin oxidase/dehydrogenase which leads to an increase in root mass, and in a portion of the genes, an increase of the line's productivity. The lower the CKX activity level, the higher the number of seeds obtained as well as higher thousand seed masses in T₀ plants as well as average root mass in T₁ plants.

### Productivity Value

In plants exhibiting a relative CKX activity value, down to 0.5 (±0.11), the number of seeds in T₀ plants grew to 142.8%; thousand seed mass to 122% and average root mass to 135.4%. In plants with a relative CKX activity level lowered to 0.69 (±0.20) the number of seeds in T₀ plants grew to 131%; thousand seed mass to 120.5% an the average root mass to 123.2%. On the basis of this data, we can assume a productivity increase under field conditions of 106-120% of the reference.

### EXAMPLE 4

### Silencing of the HvCKX2 Gene Using hpRNA/siRNA Leads to Decreased Productivity and Root Mass in Cereal Plants

This example shows that the silencing of cereal CKX genes expressed mainly in the somatic tissues (leaves) of cereal leads to an effect opposite of that claimed.

As a result of the biolistic transformation with a vector for silencing the gene HvCKX2, we obtained only 5 potentially transgenic Golden Promise plants and three Scarlett plants. The average relative CKX activity levels in the roots of T₁ seedlings of seven lines (triple analysis, 3×5 seedlings) ranged from 0.88 to 1.21 and were within the margin of error for the control plants. One of the Scarlett lines exhibited a significantly increased activity of 2.37±0.02. The productivity data for these lines encompassing seed number, thousand seed mass, the relative root mass in T₁ seedlings as well as and relative CKX enzymatic activity are shown in Table 6. The productivity of control lines in vitro as well as root mass in both control strains, Scarlett and Golden Promise, was higher than in lines transformed with the construct for silencing the expression of HvCKX2.

TABLE 6
Number of seeds and thousand seed mass (TSM) in Tₒ plants
as well as average root mass and average CKX activity in T₁ roots
of a transformed line vector silencing gene HvCKX2.

| | | | | avg. | CKX | std. |
|---|---|---|---|---|---|---|
| | | seed | TSM | root | activ- | devi- |
| | | number | (g) | mass | ity | ation |
| Golden Promise | | 1953 | 39.06 | 36.38 | | |
| 1 | II FG KP/1A | 275 | 32.86 | 41.60 | | |
| 2 | II FG KP/1B | 248 | 31.42 | 46.28 | | |
| 3 | II FG KP/1C | 198 | 32.16 | 46.00 | 1.00 | 0.00 |
| | average | 240.33 | 32.15 | 44.63 | | |
| 4 | II FG/1A | 292 | 32.22 | 30.92 | 0.93 | 0.29 |
| 5 | II FG/1 B | 292 | 30.58 | 30.08 | 0.94 | 0.20 |
| 6 | II FG/2 | 190 | 29.02 | 54.88 | 0.88 | 0.12 |
| 7 | II FG/3 | 116 | 33.04 | 48.08 | 1.08 | 0.19 |
| 8 | II CG/1 | 55 | 9.68 | 22.00 | 1.21 | 0.00 |
| | average | 189 | 26.91 | 37.19 | 1.01 | 0.16 |
| Scarlett | | 2066 | 41.32 | 41.50 | | |
| 9 | BS KP/1 | 151 | 38.52 | 41.67 | | |
| 10 | BS KP/2A | 145 | 34.2 | 32.92 | | |
| 11 | BS KP/2B | 155 | 34.62 | 31.07 | 1.00 | 0.00 |
| 12 | BS KP/2C | 229 | 28.78 | 28.75 | | |
| 13 | FS KP/1 | 215 | 29.82 | 36.25 | | |
| 14 | FS KP/2 | 251 | 31.56 | 41.67 | | |
| | average | 191 | 32.917 | 35.39 | | |
| 15 | II FS 1A | 117 | 22.92 | 28.08 | 1.19 | 0.29 |
| 16 | II FS/1 B | 307 | 26.2 | 25.00 | 1.02 | 0.34 |
| | II FS/1C | 93 | 32.1 | 36.33 | 2.37 | 0.02 |
| | average | 172.33 | 27.07 | 29.81 | 1.52 | 0.22 |

For the quantitative analysis of the expression of the HvCKX2 gene we used the following primers:

| Primer | Sequence | use |
|---|---|---|
| qCKX21 | GGCGAACTCTGGATAAATGTCTTG | RT-PCR and qRT-PCR |
| qCKX22 | AGTTCTGTTCTGGTGAGCAAGTGAC | of the HvCKX2 gene |

### EXAMPLE 5

### Analysis of the Expression of Native HvCKX1 and HvCKX2 in Various Tissues of Golden Promise and Scarlett barley strains

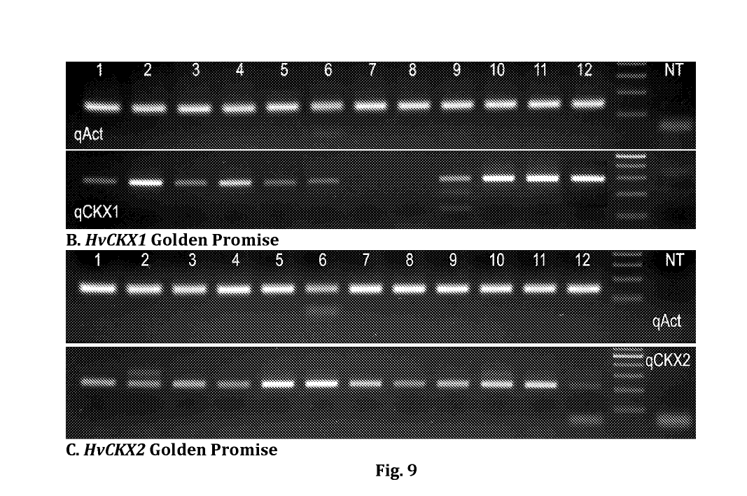

As an additional experiment we analysed the expression of the genes HvCKX1 and HvCKX2 in various tissues of control barley strains, Golden Promise and Scarlett (FIG. 9 A, B, C). Literature data on this topic are very scant and insufficient for selecting appropriate tissues for analysis and interpretation of silencing results. As is evident from FIGS. 9 A and B, the high expression of HvCKX1 in the tissues of control plants occurs in seedling roots and the inflorescences of the three studied stages, wherein it is highest in the head 7 days post pollination (7 DAP). The expression of the HvCKX2 gene is evident in all 12 examined tissues (FIG. 9 C) wherein the highest amplification was noted in the developing and developed leaf of a 2-3 week old plant.

### SUMMARY

We obtained 108 potentially transgenic lines of two strains of barley, Golden Promise and Scarlett, containing a silencing cassette against the genes HvCKX1 and HvCKX2 (as well as T-DNA without a silencing cassette as controls).
We confirmed, using CKX enzymatic activity level measurements in the root, a very sharp decrease in enzymatic activity in almost 80 of the lines (40 from 52 tested lines) of the Golden Promise strain.
We confirmed, using quantitative measurements of HvCKX1 expression, the achieved effect of silencing the expression in transgenic lines; it was positively correlated with plant productivity and root mass.
We showed that there is a strong correlation between a decreased CKX activity level and productivity (seed number and thousand seed mass) and root mass in lines with silencing.

We confirmed experimentally that the genetic modification method using a hpRNAi vector introduced via stable transformation into cereal facilitates:
silencing of the activity of particular genes of the CKX family,
function analysis,
production of culture material with novel, preferential characteristics connected with the productivity of plants relating to seed mass and number, as well as the structure of the root system.

### LITERATURE CITED

Galuszka P, Frebortova J, Werner T, Hamada M, Strnad M, Schmulling T, Frebort I. 2004. Cytokinin oxidase/dehydrogenase gene es in barley and wheat. Cloning and heterologous expression. Eur. J. Biochem. 271: 3990-4002.
Gamborg O L, Miller R A, Ojima K. 1968. Nutrient requirements of suspension cultures of soybean root cells. Exp. Celi Res. 50:151-158.
Murashige T, Skoog F. 1962. A revised medium for rapid growth and bioassays with tobacco tissue cultures. Physiol. Plant. 15: 473-497.
Przetakiewicz A, Karas A, Orczyk W, Nadolska-Orczyk A. 2004. Agrobacterium-mediated transformation of polyploid cereal. The efficiency of selection and transgene expression in wheat. Cell. Mol. Biol. Lett. 9: 903-917.
Przetakiewicz A, Orczyk W, Nadolska-Orczyk A. 2003. The effect of auxin on plant regeneration of wheat, barley and triticale. Plant Celi, Tissue Org. Cult. 73: 245-256.
Wan Y, Lemaux P G. 1994. Generation of large numbers of independently transformation fertile barley plants. Plant Physiol. 104: 37-48.

1. A DNA cassette for increasing the productivity of cereal plants and/or root mass comprising the following elements: an expression promoter, a DNA fragment from a coding or non-coding portion of the cereal CKX cytokinin oxidase gene in a sense orientation, an intron, a DNA fragment from a coding or non-coding portion of the cereal CKX cytokinin oxidase gene in an antisense orientation and a 3′ transcription terminator 3′, wherein the cereal cytokinin oxidase gene is selected from among genes expressed in the developing head and/or root from a group encompassing the genes HvCKX, TaCKX, ZmCKX, ScCKX and AsCKX.

2. A cassette according to claim 1, characterized in that the expression of CKX is depressed, and its expression leads to the formation of hpRNA, and then siRNA for silencing the expression of at least one CKX gene.

3. A cassette according to claim 1, characterized in that it contains a fragment of the CKX cytokinin oxidase gene possessing a nucleotide sequence selected from among a group encompassing: Sequence 1, Sequence 3, Sequence 4, Sequence 5 or a fragment thereof.

4. A cassette according to claim 1, characterized in that it possesses a nucleotide sequence represented as Sequence 6 or Sequence 8.

5. A binary vector containing a DNA cassette defined in claims 1 to 4.

6. A strain of A. tumefaciens containing a binary vector according to claim 5.

7. A method of obtaining a cereal plant with increased productivity, characterized in that:
a) a DNA cassette defined by claims 1 to 4 is produced, whereafter it is placed under the control of a promoter active in the modified cell, b) the resulting DNA cassette is introduced into the genome of a cereal plant, c) a cereal plant with increased productivity is derived from the resulting cell and/or d) a cereal plant with increased root mass is derived from the resulting cell.

8. A method according to claim 7, characterized in that during stage b) the transformation of the cereal plant cell is performed using a strain of A. tumefaciens containing a binary vector according to claim 5.
